(19) United States
(12) Patent Application Publication
(10) Pub. No.: US 2011/0190855 A1
(43) Pub. Date: Aug. 4, 2011

(54) Cooling Assembly

(76) Inventor: Michael Amalfi, Pittsford, NY (US)

(21) Appl. No.: 12/978,211

(22) Filed: Dec. 23, 2010

Related U.S. Application Data
(60) Provisional application No. 61/289,874, filed on Dec. 23, 2009.

(51) Int. Cl. A61F 7/10

(52) U.S. Cl. 607/112

(57) ABSTRACT

A cooling assembly is disclosed. The assembly includes a substrate and one or more cooling devices. The cooling device includes water and a reactant (such as ammonium nitrate), which when mixed, result in an endothermic reaction. One type of cooling device has an impermeable bag and a selectively breakable bag inside. Another type of cooling device has an impermeable bag and a selectively breakable barrier, which divides the bag into two packages.

In use, when the water and reactant are allowed to mix, the cooling device becomes cool. The cooled device absorbs heat from an injured person placed on the device, thereby minimizing inflammation that may occur from injuries sustained by the person. One type of substrate that is in keeping with the invention includes a body portion, a first wrap-around portion, a second wrap-around portion, and a hood. Another type of substrate that is in keeping with the invention includes a body portion, a first side cooling flap, a second side cooling flap, and a hood.

### CROSS-REFERENCE TO RELATED APPLICATION

This application claims the benefit of priority to U.S. provisional patent application Ser. No. 61/289,874, filed on Dec. 23, 2009.

### FIELD OF THE INVENTION

This invention relates to the field of emergency medical devices, including cooling devices used for therapeutic reasons in time-critical situations.

### BACKGROUND OF THE INVENTION

Great interest is currently being generated in the treatment of spinal cord injury, heart attack, stroke, head injury, and other traumatic injuries by the use of cooling to control swelling and prevent further injury. The efficacy of this treatment increases as the time between initial injury and application of the treatment decreases. Therefore, it is desirable to have cooling equipment for use at the scene of the injury in order to enable swift treatment.

### BRIEF SUMMARY OF THE INVENTION

A medical device according to the invention includes one or more cooling devices. The cooling devices may be arranged as part of an assembly. The cooling devices may be fitted into a patient support device, or otherwise applied to a patient support surface. Such an assembly may be used to prevent swelling in an injured person or to preserve the remains of a dead person.

In an embodiment of the invention, the medical device may be a litter or stretcher having a mattress, which includes a cooling device. For example, the medical device may be integrally formed with a mattress, and the mattress may be adapted to fit an emergency gurney, litter or stretcher of the types known in the art.

The cooling devices may have an impermeable bag and a selectively breakable barrier, which divides the cooling device into two storage areas. One of the storage areas may be filled with water, and the other storage area may be filled with a chemical that reacts with water to produce an endothermic reaction.

Another type of cooling device has an impermeable outer bag and a selectively breakable inner bag. The outer bag may include water and the inner bag may include a chemical which will react with the water to produce an endothermic reaction. Alternatively, the inner bag may include water and the outer bag may include the chemical. The chemical (“reactant”), such as ammonium nitrate, reacts with water to produce an endothermic reaction.

In use, the selectively breakable barrier or selectively breakable bag, as the case may be, is broken in order to allow the reactant to react with the water, thereby causing an endothermic reaction. This reaction will cool the water contained within the impermeable bag, the bag itself, and any objects in thermal communication with the impermeable bag. The selectively breakable barrier may be broken by increasing the pressure of a first storage area relative to a second storage area, or vice versa, by applying a force to one of the storage areas. Similarly, the selectively breakable barrier may be broken by increasing the pressure to the outer bag relative to the inner bag. Alternatively, pressure may be applied directly to the selectively breakable barrier.

The present invention may be embodied as a medical device for supporting a human body. The medical device may be used to transport an injured person or a dead body. In this use, the cooling assembly may include a mattress upon which the injured person is placed. The mattress may be adapted to fit an emergency gurney, litter or stretcher of the types known in the art. Multiple cooling devices may be used in this medical device.

The present invention may be directed to a medical bed for the support of an injured person. In this use, the cooling assembly may form, may be embedded within, or may encapsulate a mattress upon which the injured person is placed. The mattress may be adapted to fit a hospital bed, which may include elevating and reclining features, of the type known in the art. Multiple cooling devices may be used in this device.

### BRIEF DESCRIPTION OF THE DRAWINGS

The invention will be described further by way of non-limiting examples, with reference to the attached drawings and diagrams in which:

FIG. 1 shows an assembly according to the invention, which includes two wrap-around portions. The assembly shown in FIG. 1 can be placed on a gurney or a stretcher;

FIG. 2 shows an assembly according to the invention, which includes a hood.

FIG. 3 shows an assembly according to the invention, which has two wrap-around portions and a hood.

FIG. 4 shows an assembly according to the invention, which includes two wrap-around portions that are staggered with respect to one another.

FIG. 5 shows an assembly according to the invention, which has one wrap-around portion.

FIG. 6 shows an assembly according to the invention in which a mattress is integral with the assembly.

FIG. 7 shows an assembly according to the invention, which includes two side cooling flaps.

FIG. 8 shows an alternate form of the cooling device.

FIG. 9 shows an assembly according to the invention, which includes a wrap-around portion, two side cooling flaps, and a hood.

### DETAILED DESCRIPTION OF THE INVENTION

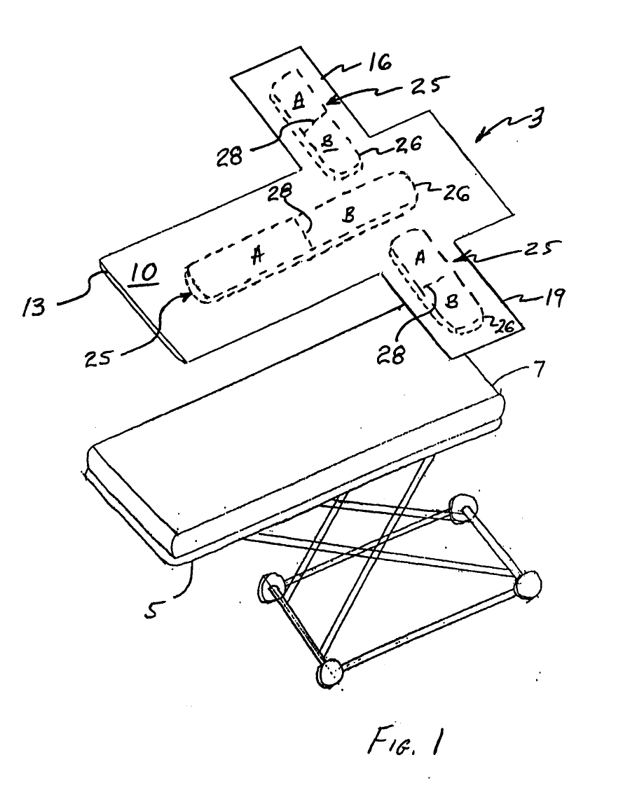

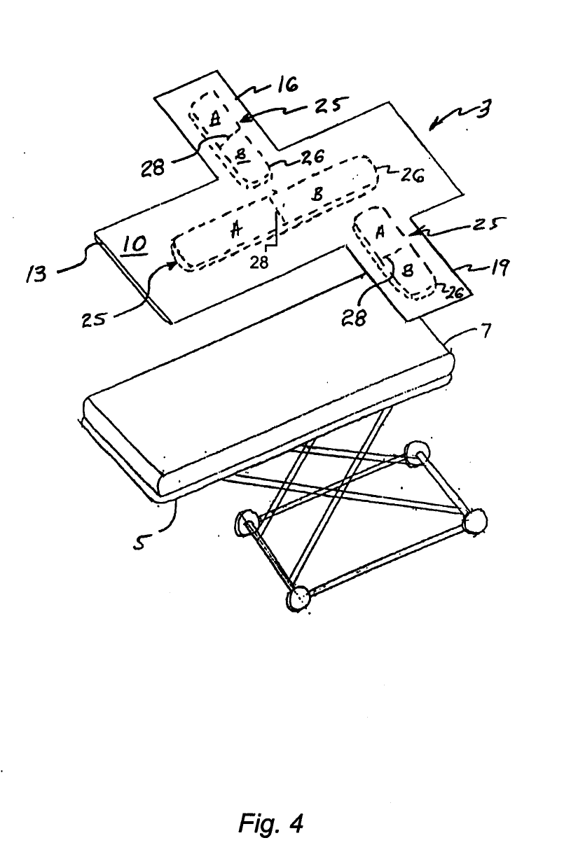

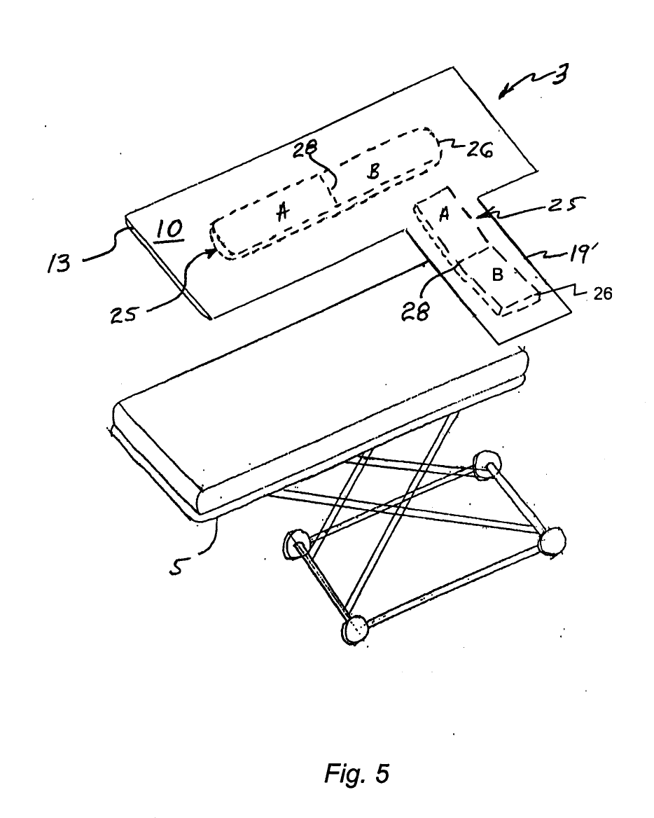

FIGS. 1 through 3 show embodiments of a cooling assembly that are in keeping with the invention. The cooling assembly 3 may be placed on a gurney 5 having a mattress 7. In FIG. 1 there is shown a cooling assembly 3 having a substrate 10, which is comprised of a body-portion 13, a first wrap-around portion 16 and a second wrap-around portion 19. The substrate 10 may be a flexible material. Attached to or integral with the body-portion 13 are one or more cooling devices which are positioned to cool a person's back when the person is lying face up on the assembly 3. The wrap-around portions 16, 19 position cooling devices, which may be used to cool a person's front torso (chest and/or abdomen) and arms by wrapping the wrap-around portions 16, 19 around the outside of a patient's arms and across their front torso. Alternatively, the wrap-around portions 16, 19 may be used to position cooling devices to cool a person's front torso by wrapping the wrap-around portions 16, 19 on the inside of the arm and around a person's front torso. It is contemplated that the wrap-around portions may be configured to meet one another, overlap one another, or may be offset from one another. For example, the wrap around portions may be configured to allow one wrap-around portion to cool a chest-portion of a person's front torso, or front torso and arms, and a second wrap-around portion to cool an abdomen-portion of a person's front torso, or front torso and arms. FIG. 4 shows an embodiment in which the wrap-around portions are offset from one another. This embodiment has the advantage that separate wrap-around portions may be used to cool distinct areas of a front torso. FIG. 5 shows an alternative embodiment of a cooling assembly having a single wrap-around portion 19′ that may be used to position cooling devices to cool a person's front torso, or front torso and arms.

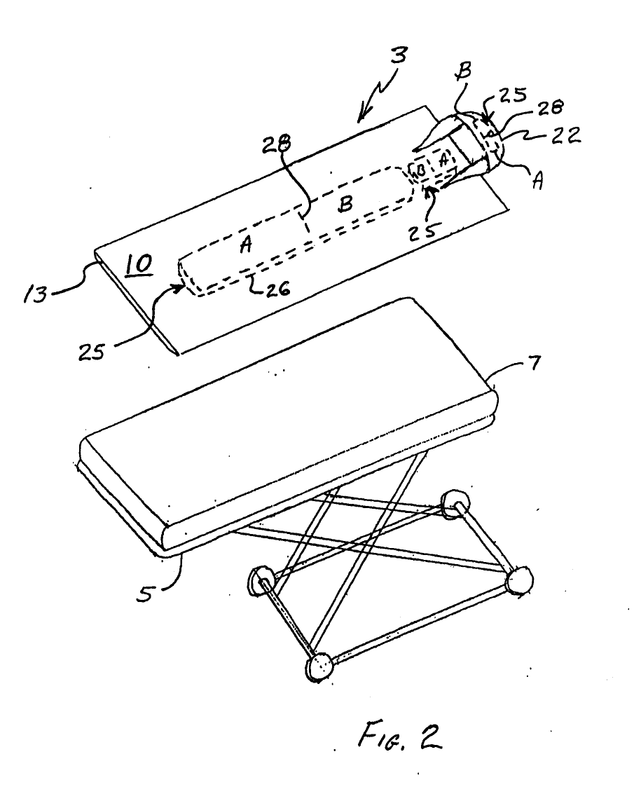

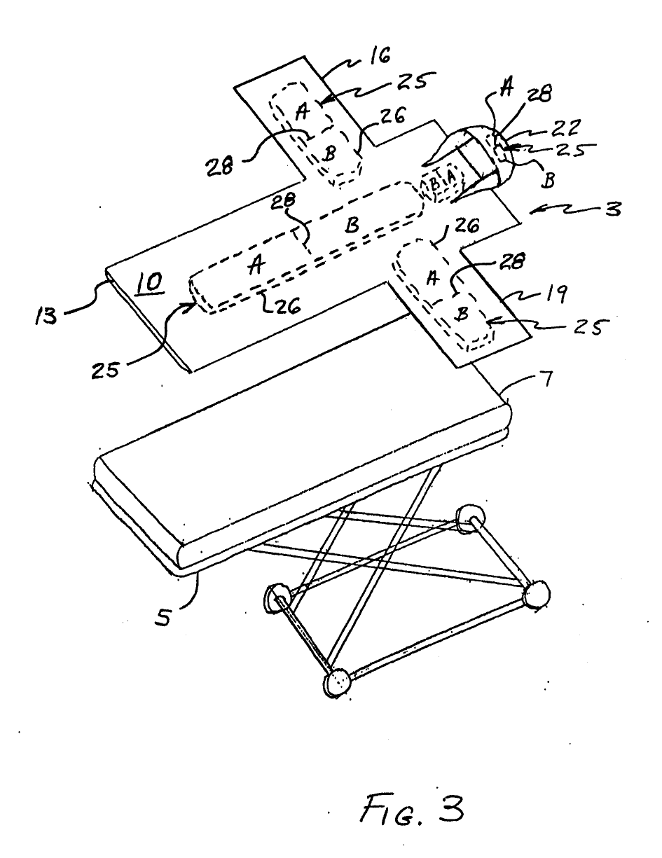

FIG. 2 shows a cooling assembly 3 having a body-portion 13 and a hood portion 22. The hood portion 22 may be used to cool a person's head. The hood portion 22 may be selectively positioned around the person's head to provide more rapid cooling of the person. FIG. 3 shows a cooling assembly 3 having a body-portion 13, a first wrap-around portion 16, a second wrap-around portion 19, and a hood portion 22.

In one or more portions of the cooling assembly 3 there may be a cooling device 25. The cooling device 25 may include an impermeable bag 26 having a selectively breakable barrier 28 therein. The barrier 28 divides the bag 26 into a first side A having water therein, and a second side B having a reactant, such as ammonium nitrate. Prior to breaking the barrier 28, the barrier 28 separates the contents of side A from the contents of side B. When the barrier 28 is broken, the contents of side A are allowed to mix with the contents of side B. By mixing the contents, an endothermic reaction allowed to occur by the reaction of the contents. This reaction will cool the contents of the cooling device 25 and any objects that are in thermal communication with the cooling device 25, including a person's body. The cooling devices may be affixed or detachably affixed to one or more portions of the cooling assembly 3 by conventional methods such as stitching, gluing, or fastening. Fasteners may include, without limitation, adhesive fastener elements, snap fastener elements, strap elements, hook-and-loop fasteners, and/or clip elements. Alternatively, the cooling devices may be housed within one or more portions of the substrate 10, or sandwiched between layers of material that comprise one or more portions of the substrate 10.

The barrier 28 may be broken before a person's body is placed on the cooling assembly 3. Alternatively, the barrier 28 may be broken after a person's body is placed on the cooling assembly 3, or at the same time a person's body is placed on the cooling assembly 3. One method of breaking the barrier 28, in order to allow the reactant to mix with the water, is to apply pressure to the barrier 28. Pressure may be applied to the barrier 28 by placement of the body on the cooling device 25 or by force applied to the barrier 28 by a user, such as a doctor, nurse, emergency medical technician, or other medical provider. The barrier may be broken by increasing the pressure of side A relative to side B, or vice versa, by applying a force to one of side A or B. Alternatively, the barrier may be broken by applying pressure directly to the barrier 28.

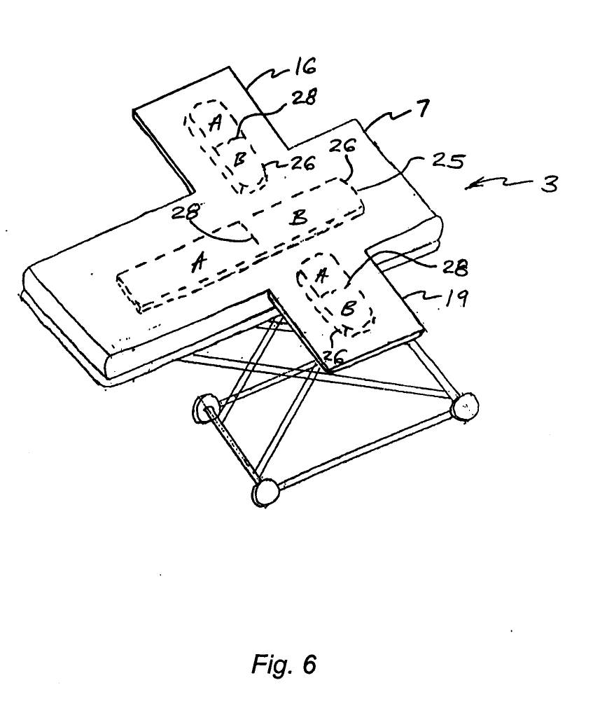

The cooling assembly 3 may be fashioned as an overlay assembly for a gurney 5, as shown in FIGS. 1 through 3. Such an overlay assembly may also be used on a litter. Alternatively, the cooling assembly 3 may be made to include a mattress 7. See FIG. 6.

The wrap-around portions 16, 19 may be made from a flexible material so that they can be laid over a person who is on the cooling assembly 3. In this manner, the person's torso and arms may be cooled. In doing so, the person may be cooled more rapidly than otherwise would be the case. A suitable flexible material includes durable fabric, such as polyester; however, alternative fabrics, or combinations of fabrics may be used. For example, an insulating layer may be attached or sandwiched between a base layer. The insulating layer may be made from an insulating fabric such as a polyester/metalized polyester blend.

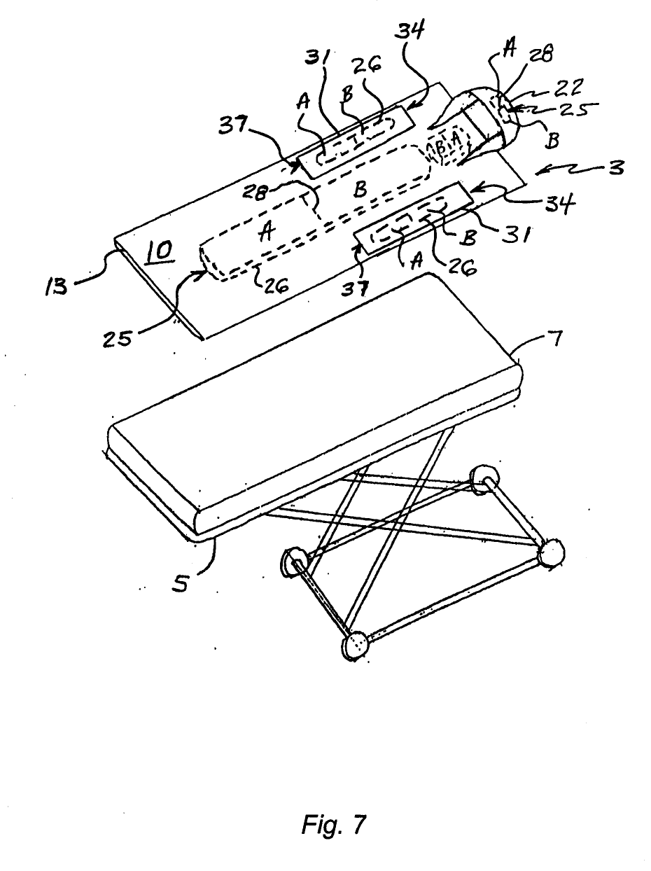

FIG. 7 depicts an embodiment of the invention which has side cooling flaps 31. The side cooling flaps 31 are shown in FIG. 7 oriented and positioned to wrap around a person's shoulder-area, thereby allowing a cooling device 25 to be placed in a person's armpit. In this manner, a person may be cooled more rapidly. A first end 34 of a side cooling flap 31 may be attached to the body-portion 13 while a second end 37 of that side cooling flap 31 is not attached to the body-portion 13. The attached end 34 may be attached to the body-portion 13 by stitching the end 34 to the body-portion 13.

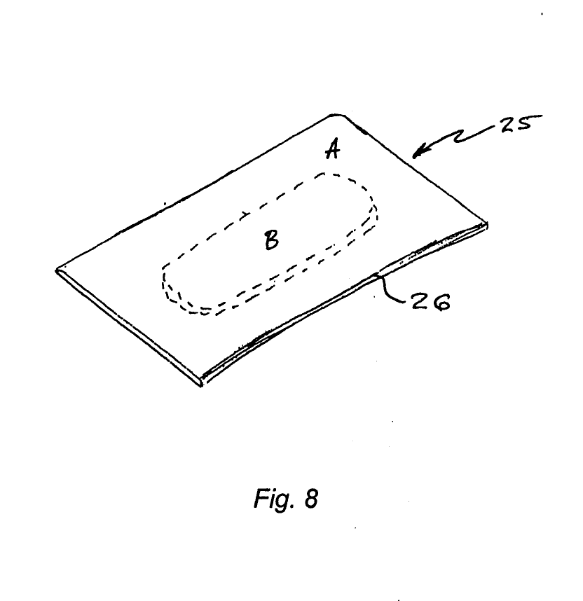

FIG. 8 depicts another type of cooling device 25. In this cooling device 25, a second package B is housed within a first package A. The second package B may be made from a material that can be broken by pressure applied to the second package B, and the first package A may be made from an impermeable material.

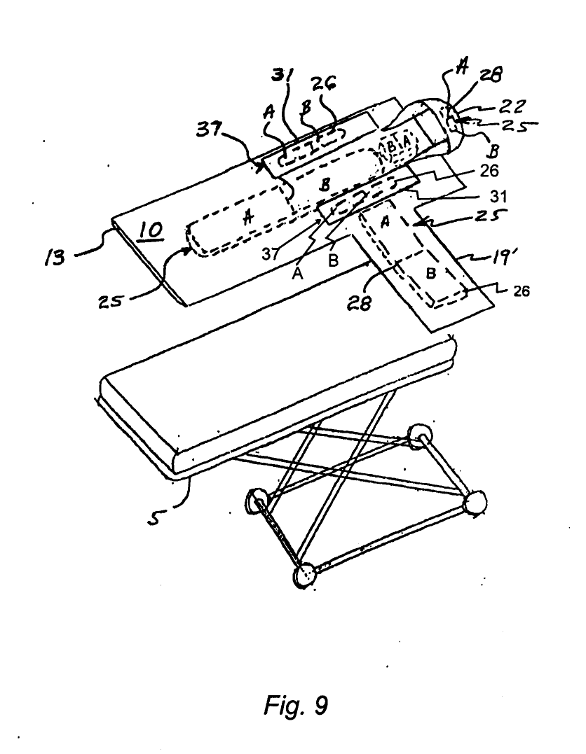

FIG. 9 shows another embodiment of the invention that has wrap-around portions 16, 19, side cooling flaps 31, and a hood portion 22. Such an embodiment may afford a medical service provider with many choices for effecting rapid cooling of the patient.

In use, a patient may be placed on a cooling assembly 3 that is sized to lay over a gurney 5. One or more cooling devices 25 may be activated on the body portion 13, for example by breaking a barrier 28 with the weight of a patient when a patient is placed on the cooling devices 25, to cause an endothermic reaction. The cooling devices 25 on the wrap around portions 16, 19, or 19′ may be activated by a medical provider prior to wrapping the wrap-around portions 16, 19, or 19′ around the patient to cool the patient's torso, or torso and arms. If the cooling assembly 3 includes side cooling flaps 31, the medical provider can wrap the side cooling flaps 31 around the patient's shoulder-area and place a cooling device 25 into the patient's armpit to cool the patient's armpit. Further, a medical provider may put a hood, which has a hood-cooling-device, on the patient's head to cool the patient's head.

Although embodiments of the invention have been described herein, the invention is not limited to such embodiments. The claims which follow are directed to the invention, and are intended to further describe the invention, but are not intended to limit the scope of the invention.

What is claimed is:

1. A cooling assembly for selectively cooling a patient, comprising:
a flexible substrate having a body portion, a first side cooling flap, a second side cooling flap, and a first wrap around portion;
a body cooling device attached to the body portion and configured with respect to the flexible substrate to cool a patient's back when the patient is lying face-up on the flexible substrate;
a first side cooling device attached to the first side cooling flap to wrap around a patient's first shoulder-area, thereby allowing a portion of the first side cooling flap to be placed in a patient's first armpit to allow cooling of the patient's first armpit;
a second side cooling device attached to the second side cooling flap to wrap around a patient's second shoulder-area, thereby allowing a portion of the second side cooling flap to be placed in a patient's second armpit to allow cooling of the patient's second armpit;
a hood portion, the hood portion having at least one hood-cooling-device to cool a patient's head;
a first torso cooling device attached to the first wrap around portion and configured with respect to the flexible substrate to wrap around a patient's torso to allow cooling of a patient's torso area.
wherein the body cooling device, the first side cooling device, the second side cooling device, and the first torso cooling device have an impermeable bag having a selectively breakable barrier therein, thereby dividing the bag into a first package and a second package; and
wherein placing a patient on the cooling assembly activates the body cooling device to cool a patient.

2. A cooling assembly for selectively cooling a patient, comprising:
a flexible substrate having a body portion and a first wrap-around portion;
a body cooling device attached to the body portion and configured with respect to the flexible substrate to cool a patient's back when the patient is supported by the flexible substrate; and
a first torso cooling device attached to the first wrap around portion and configured with respect to the flexible substrate to wrap around a patient's torso to allow cooling of a patient's torso area.

3. The cooling assembly of claim 2, further comprising a second wrap-around portion and a second torso cooling device attached to the second wrap around portion and configured with respect to the flexible substrate to wrap around a patient's torso to allow cooling of a patient's torso area.

4. The cooling assembly of claim 2, wherein the cooling assembly is shaped to fit a gurney.

5. The cooling assembly of claim 2, wherein the cooling assembly is integrally formed into a mattress.

6. The cooling assembly of claim 2, wherein the body cooling device has an impermeable bag having a selectively breakable barrier therein, thereby dividing the bag into a first side and a second side.

7. The cooling assembly of claim 6, wherein the first torso cooling device has an impermeable bag having a selectively breakable barrier therein, thereby dividing the bag into a first side and a second side.

8. The cooling assembly of claim 7, wherein the first side includes water.

9. The cooling assembly of claim 8, wherein the second side includes a reactant.

10. The cooling assembly of claim 9, wherein the reactant is ammonium nitrate.

11. The cooling assembly of claim 2, wherein the body cooling device and the first torso cooling device have a selectively breakable bag inside an impermeable bag.

12. The cooling assembly of claim 2, further comprising a hood portion, the hood portion having at least one hood-cooling-device for cooling a patient's head.

13. The cooling assembly of claim 2, wherein placing a patient on the cooling device activates the body cooling device to cool the patient.

14. A cooling assembly for selectively cooling a patient comprising:
a flexible substrate having a body portion, a first side cooling flap, and a second side cooling flap;
a body cooling device attached to the body portion and configured with respect to the flexible substrate to cool a patient's back when the patient is lying face-up on the flexible substrate;
a first side cooling device attached to the first side cooling flap to wrap around a patient's first shoulder-area, thereby allowing a portion of the first side cooling flap to be placed in a patient's first armpit to cool the patient's first armpit; and
a second side cooling device attached to the second side cooling flap to wrap around a patient's second shoulder-area, thereby allowing a portion of the second side cooling flap to be placed in a patient's second armpit to cool the patient's second armpit.

15. The cooling assembly of claim 14, further comprising a hood portion, the hood portion having at least one hood-cooling-device for cooling a patient's head.

16. The cooling assembly of claim 14, wherein the first side cooling flap comprises a fixed first end attached to the body portion and an unfastened second end for wrapping around a patient's first shoulder area, and the second side cooling flap comprises a fixed first end attached to the body portion and an unfastened second end for wrapping around a patient's second shoulder area.

17. The cooling assembly of claim 14, wherein the body cooling device, the first side cooling device, and the second side cooling device have an impermeable bag having a selectively breakable barrier therein, thereby dividing the bag into a first side and a second side.

18. The cooling assembly of claim 17, wherein the first side contains water.

19. The cooling assembly of claim 18, wherein the second side contains ammonium nitrate.

20. The cooling assembly of claim 14, wherein the body cooling device, the first side cooling device, and a second side cooling device have a selectively breakable bag inside an impermeable bag.

21. The cooling assembly of claim 14, wherein placing a patient on the cooling assembly activates the body cooling device to cool a patient.
